SEQUENCE LISTING (6 sequences)

SEQ ID NO: 1 - DNA, 4485 bp, Homo sapiens
gtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggcaagaaggctgcttgatcttgccacggtgaaagcgaatggattggctgccttccttctacaacatgttcaggaattaccagtcccattggccctgcctttggaagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtatttgcgcgataacaatatctcagaccgaggcatctgcaagctcattgaatgtgctcttcactgcgagcaattgcagaagttagctctattcaacaacaaattgactgacggctgtgcacactccatggctaagctccttgcatgcaggcagaacttcttggcattgaggctggggaataactacatcactgccgcgggagcccaagtgctggccgaggggctccgaggcaacacctccttgcagttcctgggattctggggcaacagagtgggtgacgagggggcccaggccctggctgaagccttgggtgatcaccagagcttgaggtggctcagcctggtggggaacaacattggcagtgtgggtgcccaagccttggcactgatgctggcaaagaacgtcatgctagaagaactctgcctggaggagaaccatctccaggatgaaggtgtatgttctctcgcagaaggactgaagaaaaattcaagtttgaaaatcctgaagttgtccaataactgcatcacctacctaggggcagaagccctcctgcaggcccttgaaaggaatgacaccatcctggaagtctggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaac

SEQ ID NO: 2 - DNA, 601 bp, Homo sapiens
cttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctcyggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctg

SEQ ID NO: 3 - DNA, 601 bp, Homo sapiens
ctcttgtcagtgagttcctgtccttaagggttagggctgggtagccctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggsgcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaat

SEQ ID NO: 4 - DNA, 330 bp, Homo sapiens
  misc_feature (142)..(142): n is a, c, g, or t
gactggctaactcctgcagtctctttaactggacagtttcaagaggaaaaccaagaatccttgaagctcaccattgtatcttcttttccaggttgtccaataactgcatcacctacctaggggcagaagccctcctgcaggncccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattc

SEQ ID NO: 5 - DNA, 5059 bp, Homo sapiens
ctggttctcaacttcttttgaaataatgttcatagagaaggagggctgtctgagattcgagggaaacaagctctcaggacttccggtcgccatgatggctgtgggcggtaaacgcggttagtgcaagcatctgggccatcttcaatggtaaaaaagatacagtaaagacataaataccacatttgacaaatggaaaaaaaggagtgtccagaaaagagtagcagcagtgaggaagagctgccgagacgggtatacagggagctaccctgtgtttctgagaccctttgtgacatctcacattttttccaagaagatgatgagacagaggcagagccattattgttccgtgctgttcctgagtgtcaactatctgggggggacattcccaggagacatttgctcagaagagaatcaaatagtttcctcttatgcttctaaagtctgttttgagatcgaagaagattataaaaatcgtcagtttctggggcctgaaggaaatgtggatgttgagttgattgataagagcacaaacagatacagcgtttggttccccactgctggctggtatctgtggtcagccacaggcctcggcttcctggtaagggatgaggtcacagtgacgattgcgtttggttcctggagtcagcacctggccctggacctgcagcaccatgaacagtggctggtgggcggccccttgtttgatgtcactgcagagccagaggaggctgtcgccgaaatccacctcccccacttcatctccctccaaggtgaggtggacgtctcctggtttctcgttgcccattttaagaatgaagggatggtcctggagcatccagcccgggtggagcctttctatgctgtcctggaaagccccagcttctctctgatgggcatcctgctgcggatcgccagtgggactcgcctctccatccccatcacttccaacacattgatctattatcacccccaccccgaagatattaagttccacttgtaccttgtccccagcgacgccttgctaacaaaggcgatagatgatgaggaagatcgcttccatggtgtgcgcctgcagacttcgcccccaatggaacccctgaactttggttccagttatattgtgtctaattctgctaacctgaaagtaatgcccaaggagttgaaattgtcctacaggagccctggagaaattcagcacttctcaaaattctatgctgggcagatgaaggaacccattcaacttgagattactgaaaaaagacatgggactttggtgtgggatactgaggtgaagccagtggatctccagcttgtagctgcatcagcccctcctcctttctcaggtgcagcctttgtgaaggagaaccaccggcaactccaagccaggatgggggacctgaaaggggtgctcgatgatctccaggacaatgaggttcttactgagaatgagaaggagctggtggagcaggaaaagacacggcagagcaagaatgaggccttgctgagcatggtggagaagaaaggggacctggccctggacgtgctcttcagaagcattagtgaaagggacccttacctcgtgtcctatcttagacagcagaatttgtaaaatgagtcagttaggtagtctggaagagagaatccagcgttctcattggaaatggataaacagaaatgtgatcattgatttcagtgttcaagacagaagaagactgggtaacatctatcacacaggctttcaggacagacttgtaacctggcatgtacctattgactgtatcctcatgcattttcctcaagaatgtctgaagaaggtagtaatattccttttaaattttttccaaccattgcttgatatatcactattttatccattgacatgattcttgaagacccaggataaaggacatccggataggtgtgtttatgaaggatggggcctggaaaggcaacttttcctgattaatgtgaaaaataattcctatggacactccgtttgaagtatcaccttctcataactaaaagcagaaaagctaacaaaagcttctcagctgaggacactcaaggcatacatgatgacagtcttttttttttttgtatgttaggactttaacactttatctatggctactgttattagaacaatgtaaatgtatttgctgaaagagagcacaaaaatgggagaaaatgcaaacatgagcagaaaatattttcccactggtgtgtagcctgctacaaggagttgttgggttaaatgttcatggtcaactccaaggaatactgagatgaaatgtggtaaatcaactccacagaaccaccaaaaagaaaatgagggtaattcagcttattctgagacagacattcctggcaatgtaccatacaaaaaataagccaactctgacatttggattctaccatagactctgtcattttgtagccatttcagctgtcttttgattaatgttttcgtggcacacatatttccatccttttatgtttaatctgtttaaaacaagttcctagtagacaccatctggttgagtcagttttttttatggtgtattttgaacccattctgatagtctcttttaactggaagatttcaattacttacgttaatgtaattattaatatgttaggatttatcctcagtcagccagtttgttatgtcttttctattctactgttatcacatttgtaccacttaaagtggaatctaggcactttatcaccatttagatcctattaccttttctcatctaggatatagttatcttctacataatctttctgtatcttaaaacccatcaataaattattatatattttctacttttaatcactcagaagatttaaaaaactcatgagaagagtaatctgttatgtttttccagatatttaccatttctgttgctcttccttcattattttccaaatttcgttctgcaaatttccacttcttctgatagacgttttttagttcttttagagtggttctgataggtacagattctcttattttttgcttcctctgaggacatctttttctcaccttcattctcagtgatgttttttgcttgtagtatttttagttgacattgttttctgttcagcagtttccttttagcttccgtatttcctgatgagaaatctgcagtcattcaaattgttgtttccctgtatgtagtgtgtcatttttctgtcagatttcaaggtatttatctttagtttttagccatttcattatgttggggatgagtttccttgttttattccctttggaatttgctccaattcataaatttgcagttttatgtcttttaccaaacttagaggttttcagcctaatttctaaaaatactttttattagcctgattttcatctttataggaaatagtttaagtgatgacaagttccaatagcttatatgcccagaaggccttcaaaataagaattttgaaagaatacagaaaacaaacttttatatccttctcatgtcttctactgtaaaattcatatgctttgctactctaaacctagtttgaaatcaacagtcttgagaatagatgaaaattttgatgaatagtggaattcttttaaatggaaacctcttacatgtgattttccttgccatctagaaataaaccatagtatttatgttgaatcaatcaatattatattttgtttttttcctcctcttctgagactcttattgtggaaatgttagacttttatgttttcctaaatgtccctgatattctacttatttagaacatcttttcattttttccattattctgattgggtaattttaatttgtctattttcaaatttgctggagtgttcacctgttgttgtctgtgtcgtcccactgagtgcattcaccaccttttaaattttggtcactgtatgtatcagttctaaaatttccattttgttctctatattttaaatttcttggcttatattctattttcctgcaaatgtgtcagcatttgcttgtttgagctttttttttttcaagacagggtctcaactctgttacccaggctggagtgcagtggtgcgatctcagctcactgcaacctctgcctcctggttcaagcgattattgtgcctcagcctcctgagtagctgggattacaggcatgcaccaccacagcccagctaattttttgtatttttagtagagacagagttttgctatgttggccaggctggttttgaactcctggcctcaagtgatccacccacctcagcctcccaaagtgctgggattacaggccactacacctggcacatttgagtattttttttttttttttttttttgagatggagtctcgctctgtcatctaggctggagtgcagtggtgtgatctcagctcactgcagcctctgtctcccgggctcaagcgattctcttgcctcagcctcctgagtagctaggactacaggtgcatgccaacacgcccggctaatttttttaaaaaatatttttagtagagacagggtttcaccattttggccaggatggtctcgatctcctgacctcatgatccacccgcctcggccttccaaagtgctgggattacaggcatgagccaccgtgcctggcctcatttgagtatttttataatgtctcttttaaagtctttgtcagataattccactgtacatgttattcagtgtttggtgtccactgagttgtcatttgccagacaagtggagatttttgcagctcatccttgtattctcagtagttccgatatgtaccctcgacatgtgaatgttatcttatgagactctgttttatttgtatccaacagaagatgtttattatttatttggctttctgtgaactgaggtcttaatatcagctcattttaaaagtctttgcagtggtattcggatctatcctgtgtgtgcctatgagattgggtgcagtgtatcctgttagctccattctcagggcgtttgaatgtgaattaggaccagcgcaatgaatgctcaagttggggttgggcgttagaattcataaaagtctttatatgctcag

SEQ ID NO: 6 - DNA, 1466 bp, Homo sapiens
ctcaggaccccactgtggccttcagcctcatcatcagccagtttcctagagaattaggttggttttatgtattgagtaacagcttaaccaataacccactggtcttcgattgcattgctcattgcctttttgtgtataggttctctagacacctccatggaagaaaacctcattgcttaaggtttgtttcaaaaatttctggattcattgctagtattgcataagctcattcattctcccctgagttcgatgaaaaacacccaaattcctctaattctcatgttcctctgtgatattgagacacagcgtccaatagttttccaacggaatagcttttcttacctgggaatgtcccccccagatagttgacactcaggaacagcacggawcaataatggctctgcctctgtctcatcatcttcttggaaaaaatgtgagatgtcacaaagggtctcagaaacacagggtagctccctgtataccctggaaaacaacaacagaatttttactatgaatataaggtaggtgcctgatgatagcataggctgtgcaggaagattttatgttaatagccatagactcaatattttatcttagggaagtcattcctcaggcccctacgactccatctcacctctcagactcccatgactctttcttacatctcattatgttaaatttaactggctctctgtttcccactatatgctgctctttccatcctaggaagcagacgtcagtcagttctcaacatctagcatttgccacaaacattggtttcataataggtcaacaagtatgttgacctatataaccttgctaagaattttagggaaaggatgagattcctaatttgtagtctcccttcatccataattggtgcccgagagaataggaccctaaaatgattgggattgcagggcattagtgagattgggcatgttttataagaacccatggaacagttatctcctcttctcccttctgcctgcaaatggtgagaggggttgcataaagcaacaaaaatgctcacagaaaaagaaaattatggatattgtacacactttcttttcccatcaaggatccttattcagatatggaacatgagagtcctatgctagatccttttctcttcttcatttttgaaggcttggtgctgtcctcctatggctggcaggaatcaagattgaggttaggagtgatggagtgtcctttatgccaagatattcaatggccaatatgacagccactagccacacctgcctatttacatttagttttaaattgttaaatgtgaaaatcagttcctcctttgaagtagccatatttcaagtgctcaaaagccacacgtggctcttgcctgccaccatgtaagacatgcctttgctcctcctttgacttctgccatgatggtgaggcctccccagccacgtgaaactaaaagaatttttctgggtaatggacat

(19) United States
(12) Patent Application Publication
(10) Pub. No.: US 2010/0015156 A1
(43) Pub. Date: Jan. 21, 2010

(54) DIAGNOSIS OF INFLAMMATORY BOWEL DISEASE IN CHILDREN

(75) Inventors: Marla Dubinsky, Los Angeles, CA (US); Stephan R. Targan, Santa Monica, CA (US); Jerome I. Rotter, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/529,106

(22) PCT Filed: Mar. 6, 2008
(86) PCT No.: PCT/US08/56103

§ 371 Date: Aug. 28, 2009

Related U.S. Application Data
(60) Provisional application No. 60/893,308, filed on Mar. 6, 2007.

(51) Int. Cl. A61K 39/395; C12Q 1/68; A61P 1/00; A61P 29/00; A61P 37/00

(52) U.S. Cl. 424/139.1; 435/6

(57) ABSTRACT

This invention provides methods of diagnosing and predicting disease progression of Crohn's disease. In one embodiment, a method of the invention is practiced by determining the presence or absence of CARD15 variants R702W, G908R, and/or 1007insC in a pediatric individual. In another embodiment, a method of the invention is practiced by determining the presence or absence of anti-Cbir1, anti-OmpC, ASCA, and/or pANCA in a pediatric individual

### FIELD OF THE INVENTION

The invention relates generally to the fields of inflammation and autoimmunity and autoimmune disease and, more specifically, to methods for diagnosing and predicting disease progression of Crohn's disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other markers, genes, allelic variants and/or haplotypes that may assist in explaining the genetic risk, predicting disease progression, diagnosing, and/or predicting susceptibility for or protection against inflammatory bowel disease including but not limited to CD and/or UC.

### SUMMARY OF THE INVENTION

Various embodiments provide methods of diagnosing susceptibility to a subtype of Crohn's Disease in a child, comprising determining the presence or absence of at least one risk variant at the CARD15 locus selected from the group consisting of SNP8, SNP12, and SNP13, and determining the presence or absence of at least one risk serological marker, selected from the group consisting of Cbir1, OmpC, and ASCA, where the presence of at least one variant and at least one risk serological marker is diagnostic of susceptibility to the subtype of Crohn's Disease in a child. In another embodiment, the subtype of Crohn's Disease in a child comprises an aggressive complicating phenotype, a small bowel disease phenotype, and/or an internal penetrating and/or fibrostenosing disease phenotype. In another embodiment, the presence of three of the risk serological markers presents a greater susceptibility than the presence of two, one or none of the risk serological markers, and the presence of two of the risk serological markers presents a greater susceptibility than the presence of one or none of the risk serological markers but less than the presence of three of the risk serological markers, and the presence of one of the risk serological markers presents a greater susceptibility than the presence of none of the risk serological markers but less than the presence of three or two of the risk serological markers. In another embodiment, the SNP8 comprises SEQ. ID. NO.: 2. In another embodiment, the SNP12 comprises SEQ. ID. NO.: 3. And in another embodiment, the SNP13 comprises SEQ. ID. NO. 4.

Other embodiments provide for methods of diagnosing susceptibility to a subtype of Crohn's Disease in a child, comprising determining the presence or absence of a high immune reactivity relative to a healthy individual for at least one risk serological marker, selected from the group consisting of Cbir1, OmpC, ASCA, 12, and pANCA, where the presence of a high immune reactivity relative to a healthy individual to at least one risk serological marker is diagnostic of susceptibility to the subtype of Crohn's Disease in a child. In another embodiment, the subtype of Crohn's Disease in a child comprises an aggressive complicating phenotype. In another embodiment, a high immune reactivity comprises a high magnitude of expression for the risk serological marker. In another embodiment, the presence of four of the risk serological markers presents a greater susceptibility than the presence of three, two, one or none of the risk serological markers, and the presence of three of the risk serological markers presents a greater susceptibility than the presence of two, one or none of the risk serological markers but less than the presence of four of the risk serological markers, and the presence of two of the risk serological markers presents a greater susceptibility than the presence of one or none of the risk serological markers but less than the presence of four or three of the risk serological markers, and the presence of one of the risk serological markers presents a greater susceptibility than the presence of none of the risk serological markers but less than the presence of four or three or two of the risk serological markers.

Various embodiments also provide methods of treating Crohn's Disease in a child, comprising determining the presence of a high immune reactivity to a risk serological marker relative to a healthy individual, and administering a therapeutically effective amount of Crohn's Disease treatment.

Other embodiments provide methods of diagnosing ulcerative colitis in an individual, comprising determining the presence or absence of a risk variant at the CAR D8 locus, where the presence of the risk variant at the CARD8 locus is diagnostic of susceptibility to ulcerative colitis. In other embodiments, the risk variant at the CARD8 locus comprises SEQ. ID. NO.: 6. In other embodiments, the individual is a child.

Various embodiments provide methods of determining the prognosis of Crohn's Disease in an individual, comprising determining the presence or absence of a high immune reactivity relative to a healthy individual for at least one risk serological marker, selected from the group consisting of Cbir1, OmpC, ASCA, and pANCA, where the presence of a high immune reactivity relative to a healthy individual to at least one risk serological marker is indicative of a prognosis of an aggressive form of Crohn's Disease. In other embodiments, the individual is a child. In other embodiments, the prognosis of an aggressive form of Crohn's Disease further comprises a rapid complicating internal penetrating and/or fibrostenosing disease phenotype.

Other embodiments provide methods of determining the prognosis of Crohn's Disease in a pediatric subject, comprising determining the presence or absence of a high immune reactivity of Cbir1, OmpC, ASCA, and pANCA in the pediatric subject relative to a child who has and maintains a non-aggressive form of Crohn's Disease, where the presence of the high immune reactivity relative to a child who has and maintains a non-aggressive Crohn's Disease is indicative of a prognosis of an aggressive form of Crohn's Disease in the pediatric subject. In other embodiments, the aggressive form of Crohn's Disease further comprises a rapid complicating internal penetrating and/or stricturing disease phenotype.

Other embodiments provide methods of treating an aggressive form of Crohn's Disease in a pediatric subject, comprising determining the presence of a high immune reactivity of Cbir1, OmpC, ASCA and pANCA relative to a child who has and maintains a non-aggressive form of Crohn's Disease to prognose the aggressive form of Crohn's Disease, and treating the aggressive form of Crohn's Disease.

Other embodiments provide methods of determining the prognosis of Crohn's Disease in a subject, comprising determining the presence or absence of a high immune reactivity in the subject relative to an individual who has and maintains a non-aggressive form of Crohn's Disease for at least one risk serological marker, selected from the group consisting of Cbir1, OmpC, ASCA, and pANCA, where the presence of the high immune reactivity relative to an individual who has and maintains a non-aggressive form of Crohn's Disease is indicative of a prognosis of an aggressive form of Crohn's Disease. In other embodiments, the subject is a pediatric subject. In other embodiments, the individual who has and maintains a non-aggressive form of Crohn's Disease is a child. In other embodiments, the aggressive form of Crohn's Disease further comprises a rapid complicating internal penetrating and/or fibrostenosing disease phenotype.

Various embodiments also provide methods of treating an aggressive form of Crohn's Disease in a subject, comprising determining the presence of a high immune reactivity relative to an individual who has and maintains a non-aggressive form of Crohn's Disease to prognose the aggressive form of Crohn's Disease, and treating the aggressive form of Crohn's Disease. In other embodiments, the subject is a pediatric subject. In other embodiments, the individual who has and maintains a non-aggressive form of Crohn's Disease is a child. In other embodiments, the aggressive form of Crohn's Disease further comprises a rapid complicating internal penetrating and/or fibrostenosing disease phenotype.

Other features and advantages of the invention will become apparent from the following detailed description, taken in conjunction with the accompanying drawing, which illustrate, by way of example, various embodiments of the invention.

### BRIEF DESCRIPTION OF THE FIGURES

FIG. 1 depicts Kaplan-Meier survival analysis. Comparison of time to progression from noncomplicating to complicating disease behaviors between patients positive for 21 immune response to ASCA, 12, and OmpC (n=97) ( - - - ) and those negative for all three (n=70) (—).

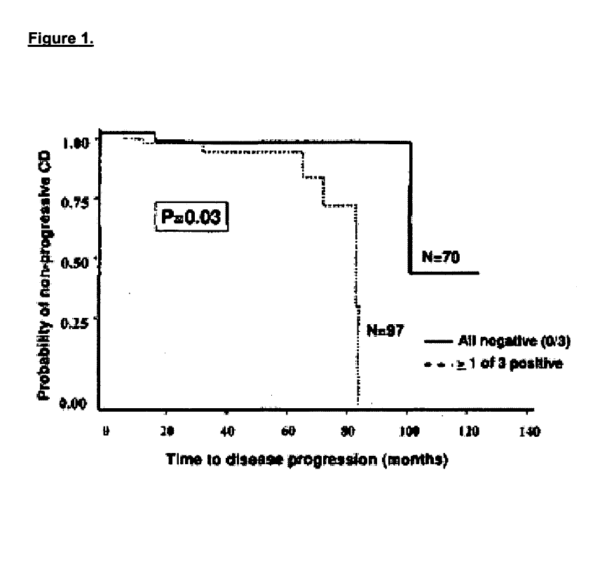

FIG. 2 depicts results of patient demographics from 796 well characterized pediatric Crohn's Disease patients as part of a study that demonstrates an increased immune reactivity predicts aggressive complicating Crohn's Disease in children.

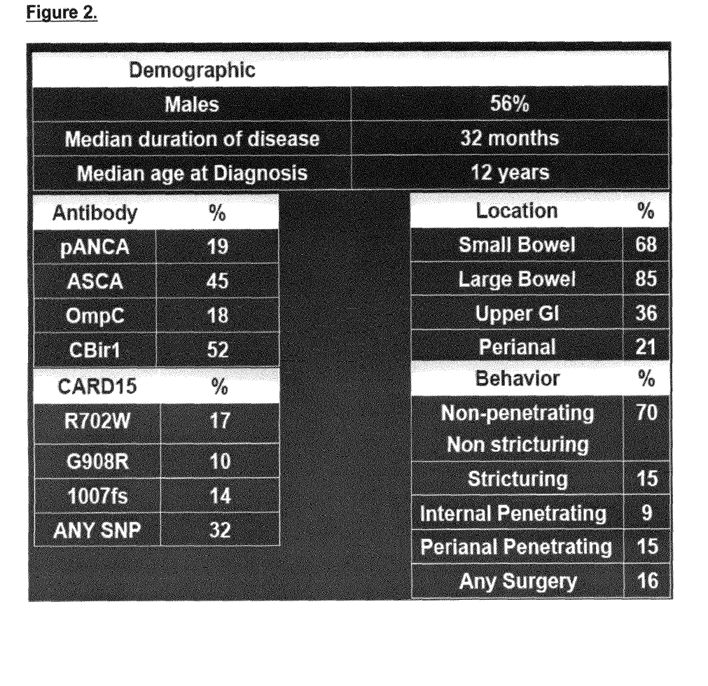

FIG. 3 depicts results demonstrating an association of immune reactivity and CARD15 with disease location through univariate analysis.

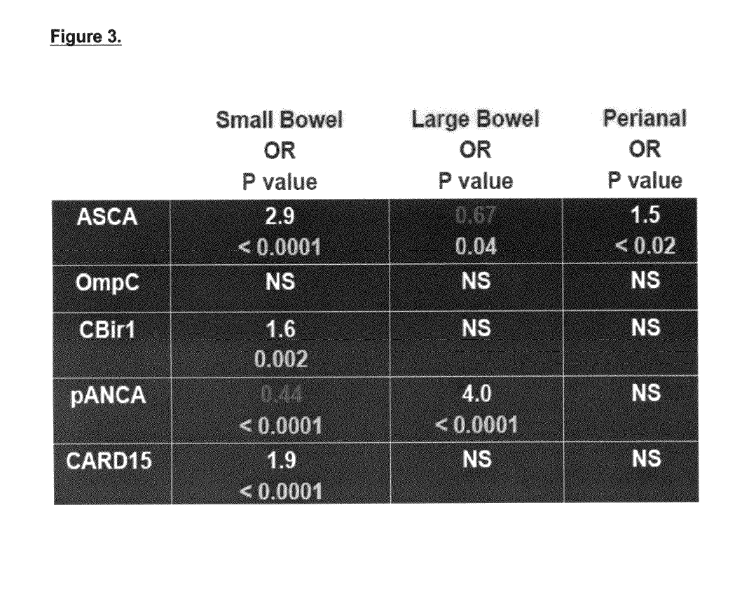

FIG. 4 depicts results demonstrating an association of immune reactivity and CARD15 with disease behavior through univariate analysis.

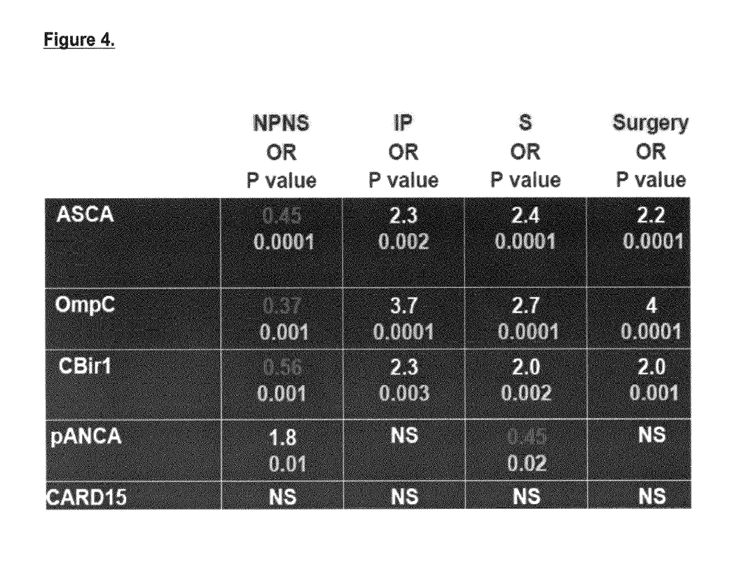

FIG. 5 depicts a chart of antibody sum and disease behavior.

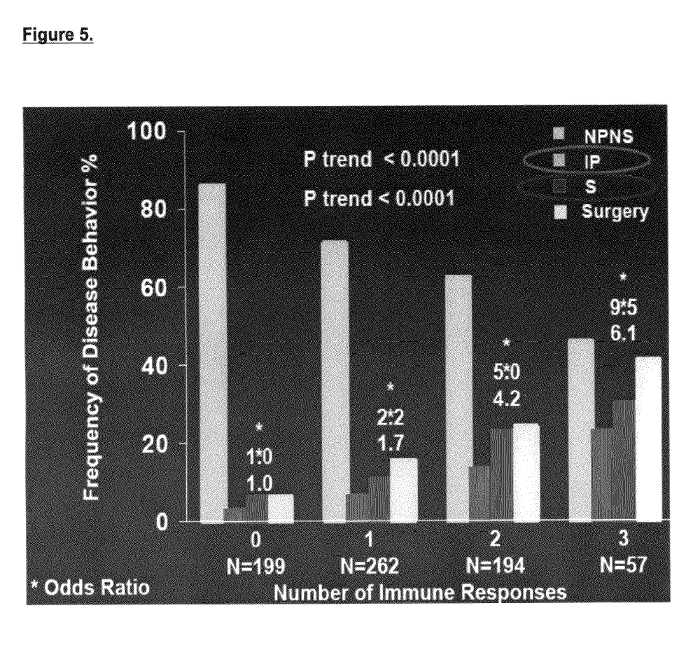

FIG. 6 depicts a chart of quartile sum and stricturing disease.

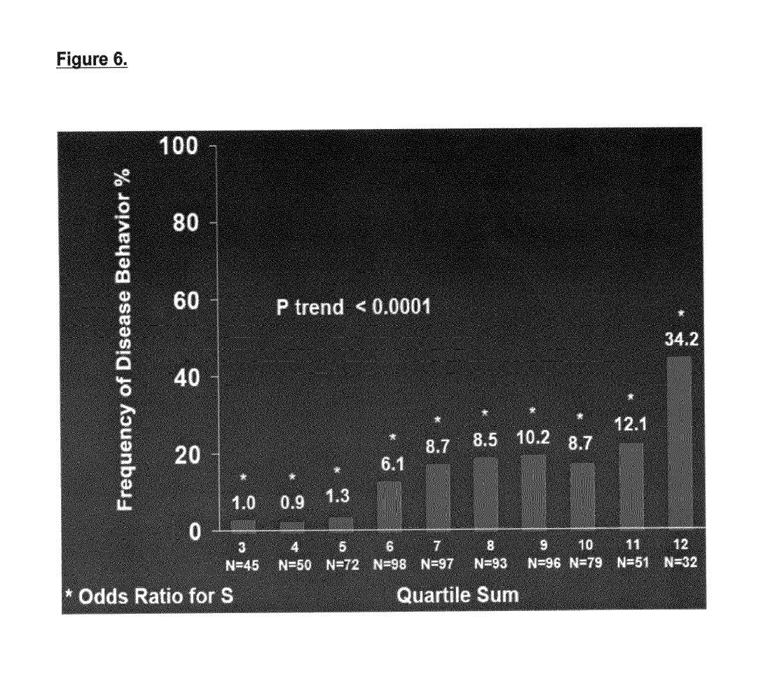

FIG. 7 depicts a chart of quartile sum groups and disease behavior.

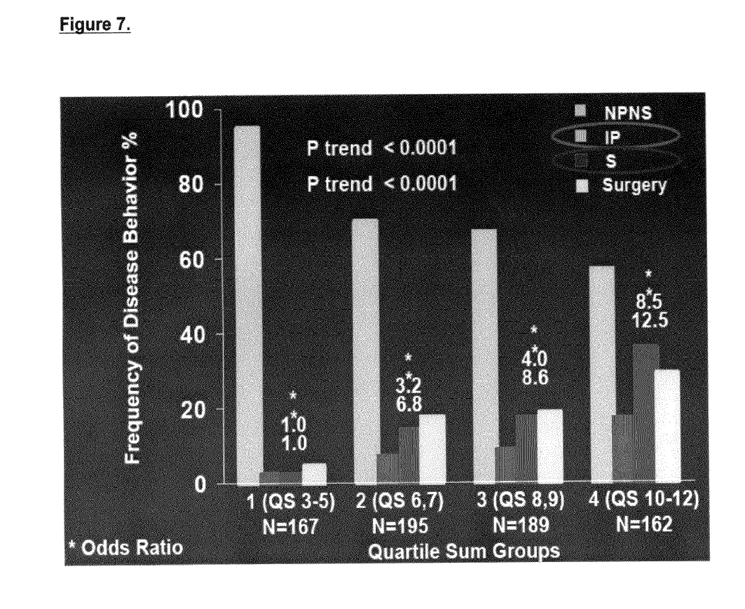

FIG. 8 depicts results demonstrating an association of immune reactivity with disease behavior using multivariate analysis.

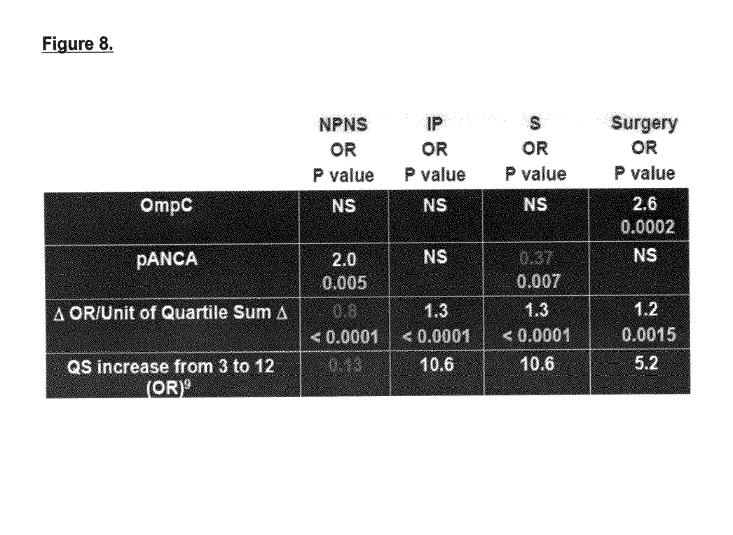

FIG. 9 depicts a chart demonstrating predictors of disease progression. The chart describes antibody sum and disease progression.

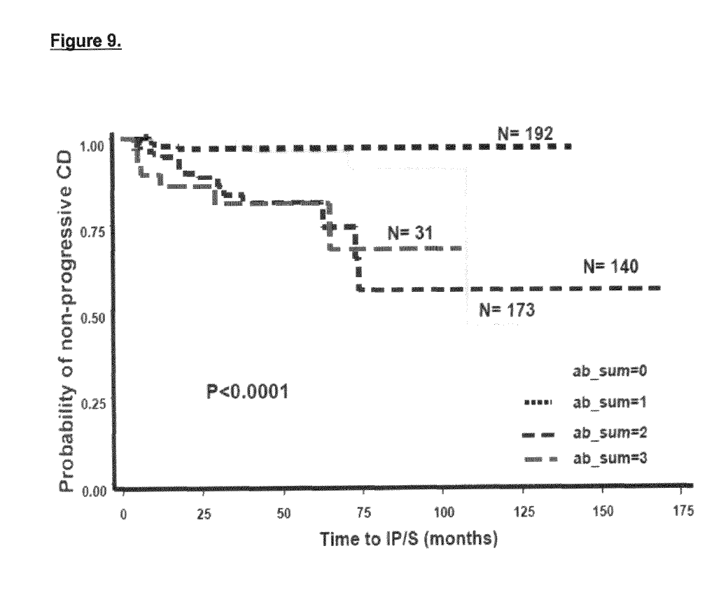

FIG. 10 depicts a chart describing predictors of disease progression. The chart describes quartile sum groups and disease progression.

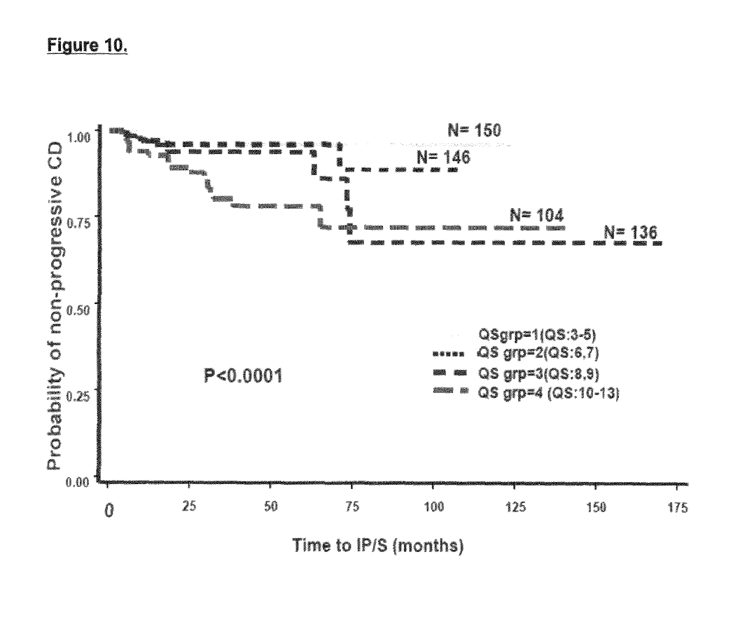

FIG. 11 depicts a chart describing predictors of disease progression. The chart describes antibody sum and surgery.

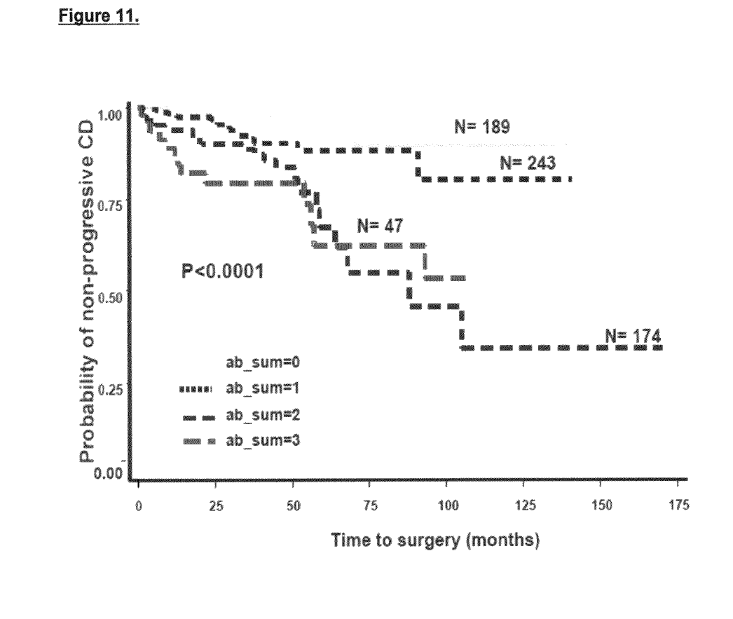

FIG. 12 depicts a chart describing hazard ratios, with immune response prediction of complications and surgery.

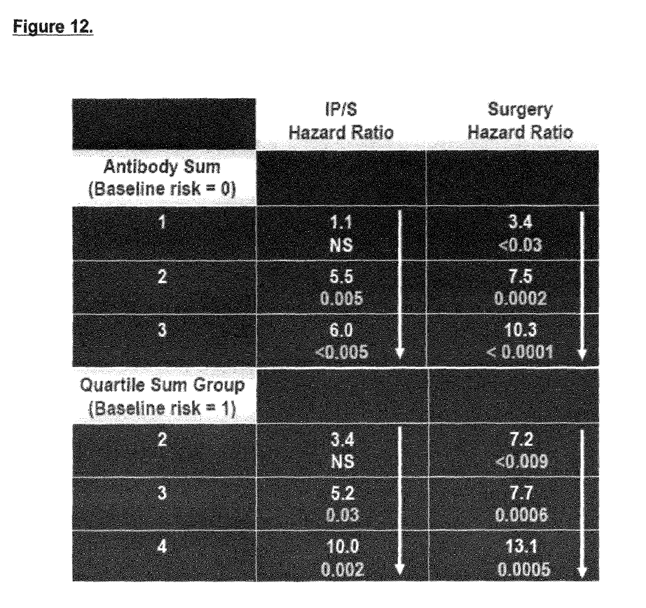

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Risk variant” as used herein refers to an allele whose presence is associated with an increase in susceptibility to an inflammatory bowel disease, including but not limited to Crohn's Disease and ulcerative colitis, relative to a healthy individual.

“Risk serological marker” as used herein refers to a serological marker whose expression is associated with an increase in susceptibility to and/or risk for rapid disease progression of inflammatory bowel disease, including but not limited to Crohn's Disease and ulcerative colitis, relative to a healthy individual.

As used herein, “antibody sum (AS)” means the number of positive antibodies per individual, such as 0, or 1 or 2, or 3 positive.

As used herein, “antibody quartile score” means the quartile score for each antibody level (<25%=1, 25-50%=2, 51%-<75%=3, 75%-100%=4).

As used herein, “quartile sum score (QSS)” means the sum of quartiles score for all of the antibodies.

As described herein, the inventors regrouped patients based on a range of quartile sum scores, defined as “Quartile Sum Score (QSS) Group.” For example, quartile sum score 3-5=group 1, 6-7=group 2, 8-9=group 3 and 10-12=group 4.

As used herein, “ASCA” means anti-Saccharomyces cerevisiae antibodies.

As used herein, “pANCA” means perinuclear anti-neutrophil cytoplasmic antibodies.

As used herein, “OmpC” means outer membrane protein C.

As used herein, “I2” means Pseudomonas fluorescens-associated sequence.

As used herein, “OR” is an abbreviation for odds ratio.

As used herein, “CI” is an abbreviation for confidence interval.

As used herein, “OCTN” is an abbreviation for organic cation transporter.

As used herein, “IP” is an abbreviation for internal penetrating disease.

As used herein, “S” is an abbreviation of stricturing disease.

As used herein, “NPNS” is an abbreviation of non-penetrating, non-stricturing disease.

As used herein, “PP” is an abbreviation of perianal penetrating.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As used herein, “CARD15” also means NOD2. As disclosed herein, an example of CARD15 is described as SEQ. ID. NO.: 1.

As used herein, SNP 8, 12, and 13, are also described as R702W, G908R, and 1007fs, respectively, as well as R675W, G881R, and 3020insC, respectively. Examples of SNP 8, 12, and 13, are described herein as SEQ. ID. NO.: 2, SEQ. ID. NO.: 3, SEQ. ID. NO.: 4, respectively.

An example of CARD8 is described herein as SEQ. ID. NO.: 5.

An example of T10C variant at the CARD8 locus is described herein as SEQ. ID. NO.: 6.

As known to one of ordinary skill in the art, there are presently various treatments and therapies available for those diagnosed with Inflammatory Bowel Disease, including but not limited to surgery, anti-inflammatory medications, steroids, and immunosuppressants.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to I2, OmpC, and Cbir. The detection of protective and risk SNPs and/or haplotypes may be used to identify at risk individuals, predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and ulcerative colitis. Other embodiments provide for methods of prognosing inflammatory bowel disease including but not limited to Crohn's Disease and ulcerative colitis. Other embodiments provide for methods of treating inflammatory bowel disease including but not limited to Crohn's Disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

### Increased Immune Reactivity Predicts Aggressive Complicating Crohn's Disease in Children

As disclosed herein, the inventors examined the association of serological immune responses and CARD15 with CD phenotype in a large well-characterized pediatric collaborative cohort. Sera were collected from 797 prospectively followed pediatric CD cases and tested for immune responses to microbial antigens: anti-Cbir1 (flagellin), anti-outer membrane protein C (anti-OmpC) and anti-Saccharomyces-cerevisiae (ASCA) using ELISA. Genotyping (TaqmanMGB) was performed for 3 CD-associated variants of CARD15 (SNPs 8, 12, 13). Disease phenotypes were determined blinded to genotype and immune responses. Associations between immune responses, CARD15 and clinical phenotype were evaluated.

As used herein, SNP's 8, 12, 13 are also referred to as R702W, G908R, 1007insC.

As further disclosed herein, CARD15 variants and immune responses were present in 34% and 78%, respectively. Small bowel (SB) location, IP and/or FS disease behavior were present in 68% (n=542) and 20% (n=152) of children after a median follow-up of 31 months. The odds of developing IP and/or FS disease were highest in patients positive for all 3 immune responses. The highest level for each individual antibody was associated with IP and/or FS with the odds being highest when using the sum of all immune response levels. Multivariate analysis confirmed the Anti-OmpC (p<0.0002) and anti-Cbir1 (p=0.005) association with IP as well as ASCA (p=0.02) and anti-Cbir1 (p=0.04) with FS. CARD15 was associated with small bowel disease (OR=1.7; p<0.0001) only, not with disease behavior.

As further disclosed herein, the rate of complicated CD increases in children as the number and magnitude of immune reactivity increases. Baseline immune response assessment may identify children at risk for complicating IP/FS phenotypes, for which early, aggressive immunomodulatory therapy could be of benefit.

In one embodiment, the present invention provides methods of diagnosing and/or predicting susceptibility to a subtype of Crohn's Disease in an individual by determining the presence or absence of immune reactivity in the individual, where the presence of immune reactivity is diagnostic of the subtype of Crohn's Disease. In another embodiment, the present invention provides methods of prognosis of Crohn's Disease in an individual by determining the presence or absence of immune reactivity, wherein the presence of immune reactivity is indicative of a complicating Crohn's Disease prognosis. In another embodiment, the present invention provides methods of treatment of Crohn's Disease by administering a therapeutically effective amount of Crohn's Disease treatment wherein there is a presence of immune reactivity in the individual. In another embodiment, the subtype is complicating Crohn's Disease. In another embodiment, the subtype is small bowel disease, internal penetrating and/or fibrostenosing. In another embodiment, immune reactivity is a high expression of ASCA, OmpC, and/or Cbir1, relative to levels found in a healthy individual. In another embodiment, the individual is a child.

In one embodiment, the present invention provides a method of diagnosing susceptibility to a subytpe of Crohn's Disease by determining the presence of immune reactivity, and determining the presence of CARD15 variants, wherein the presence of immune reactivity and one or more CARD15 variants is diagnostic of susceptibility to the subtype of Crohn's Disease. In another embodiment, the present invention provides a method of prognosis of Crohn's Disease in an individual by determining the presence of immune reactivity, and determining the presence of CARD15 variants, wherein the presence of immune reactivity and one or more CARD15 variants is indicative of a complicating Crohn's Disease prognosis. In another embodiment, the present invention provides a method of treatment of Crohn's Disease by administering a therapeutically effective amount of Crohn's Disease treatment wherein there is a presence of immune reactivity and CARD15 variants in the individual. In another embodiment, the CARD15 variants comprise SNPs 8, 12, and/or 13. In another embodiment, immune reactivity is a high expression of ASCA, OmpC, and/or Cbir1, relative to levels found in a healthy individual. In another embodiment, the individual is a child. In another embodiment, the subtype of Crohn's Disease is small bowel disease, internal penetrating and/or fibrostenosis.

### Serum Immune Responses Predict Rapid Disease Progression Among Children with Crohn's Disease Immune Responses Predict Disease Progression

As disclosed herein, sera were collected from 196 pediatric CD cases and tested for immune responses: anti-I2, anti-outer membrane protein C (anti-OmpC), anti-CBir1 flagellin (anti-CBir1), and anti-Saccharomyces-cerevisiae (ASCA) using ELISA. Associations between Immune responses and clinical phenotype were evaluated. Fifty-eight patients (28%) developed internal penetrating and/or stricturing (IP/S) disease after a median follow-up of 18 months. Both anti-OmpC (p<0.0006) and anti-12 (p<0.003) were associated with IP/S disease. The frequency of IP/S disease increased with increasing number of immune responses (p trend=0.002). The odds of developing IP/S disease were highest in patients positive for all four immune responses (OR (95% CI): 11 (1.5-80.4); p=0.03). Pediatric CD patients positive for ≧1 immune response progressed to IP/S disease sooner after diagnosis as compared to those negative for all immune responses (p<0.03).

As further disclosed herein, the presence and magnitude of immune responses to microbial antigens are significantly associated with more aggressive disease phenotypes among children with CO. This demonstrates that the time to develop a disease complication in children is significantly faster in the presence of immune reactivity, thereby predicting disease progression to more aggressive disease phenotypes among pediatric CD patients.

In one embodiment, the present invention provides methods of diagnosing and/or predicting susceptibility to a subtype of Crohn's Disease in an individual by determining the presence or absence of immune reactivity in the individual, where the presence of immune reactivity is diagnostic of the subtype of Crohn's Disease. In another embodiment, the present invention provides methods of prognosis of Crohn's Disease in an individual by determining the presence or absence of immune reactivity, wherein the presence of immune reactivity is indicative of a complicating Crohn's Disease prognosis. In another embodiment, the present invention provides methods of treatment of Crohn's Disease by administering a therapeutically effective amount of Crohn's Disease treatment wherein there is a presence of immune reactivity in the individual. In another embodiment, the subtype is complicating Crohn's Disease. In another embodiment, the subtype is small bowel disease, internal penetrating and/or fibrostenosing. In another embodiment, immune reactivity is a high expression of ASCA, OmpC, Cbir1, and/or 12 relative to levels found in a healthy individual. In another embodiment, the individual is a child.

### CARD8: A Novel Association with Childhood-Onset Ulcerative Colitis (UC)

As disclosed herein, the inventors investigated the association of the CARD8-T10C polymorphism with susceptibility to UC and CD in children. DNA was collected from 342 subjects (75 CD trios, 39 UC trios). Both parents and the affected child were genotyped for 3 allelic variants of the CARD15 gene (R702W. G908R, 1007insC, also referred to as SNP 8, 12 and 13) as an association control and 1 variant of the CARD gene (T10C) using Taqman technology. The transmission disequilibrium test (TDT) was used to test association with either UC or CD using GENEHUNTER 2.0.

As further disclosed herein, CARD8 allele T was present in 63% of CD patients and 77% of UC patients. CARD15 frequency (any variant) was 25% and 11% in CD and in UC, respectively. Similar frequencies were observed for parents for both genes. As expected, transmission distortion was seen for all CARD15 variants in CD, but not in UC. No association was observed between CARD8 and CD, however, in contrast, TDT showed a highly significant association with UC, with over transmission of the CARD8 common allele.

As further disclosed herein, this shows a CARD8 association with childhood-onset UC. The over transmission of the common allele in this analysis is similar to that which is seen with PPARgamma in type 2 diabetes and the insulin gene polymorphism in type 1 diabetes. These findings are in contrast to the adult CD association showing different mechanisms for pediatric IBD.

In one embodiment, the present invention provides methods of diagnosing and/or predicting susceptibility to ulcerative colitis in an individual by determining the presence or absence of a CARD8 risk variant in the individual, where the presence of the CARD8 risk variant is diagnostic of ulcerative colitis. In another embodiment, the present invention provides methods of treatment of ulcerative colitis by administering a therapeutically effective amount of ulcerative colitis treatment wherein there is a presence of a CARD8 risk variant in the individual. In another embodiment, the CARD8 variant is T10C. In another embodiment, the individual is a child.

### Antibodies to a Novel Flagellin (Cbir1) Adds Clinical Utility to the Diagnosis and Differentiation of Pediatric IBD

As disclosed herein, sera from 331 pediatric IBD patients (111 UC, 220 CD) were tested by ELISA for anti-OmpC, anti-12, ASCA, anti-Cbir1 and pANCA. Quantitative and qualitative expression of antibody markers was evaluated. Anti-Cbir1 was present in 55% of CD vs. 15% of UC (p<0.001). 41% of anti-Cbir1 (+) UC patients were also positive for >I CD-related antibody. Anti-Cbir1 was present in 53% of ASCA(−) CD patients and in 52% (31/60) of patients negative for all antibodies. The most Cbir1 reactive CD subset was OmpC+/12+(74% median=49) and least reactive was ASCA+ (56%, median=31). 13.5% of pANCA (+) only UC patients were anti-CBir1 (+) as compared to 35% of pANCA(+) only CD patients (p=0.03). Both pANCA and anti-Cbir1 levels were higher in pANCA (+) CD vs. UC (median pANCA: 46.6 vs. 70.0: p=0.003, and median anti-Cbir1: 21 vs. 12 p<0.0001).

As further disclosed herein, anti-Cbir1 increased detection of CD cases negative for all other antibodies. Cbir1 reactivity added to the differentiation of pANCA+ CD from pANCA+ UC and can minimize misdiagnosed CD colitis patients. Both the presence and magnitude of anti-Cbir1 reactivity adds to the clinical utility of presently known antibodies in pediatric IBD.

In one embodiment, the present invention provides methods of diagnosing and/or predicting susceptibility to inflammatory bowel disease in a child by determining the presence or absence of high expression of anti-Cbir1 relative to a healthy individual, wherein the presence of the high expression of anti-Cbir1 relative to a healthy individual is indicative of susceptibility to inflammatory bowel disease in the child. In another embodiment, the present invention provides methods of treatment for inflammatory bowel disease in a child by administering a therapeutically effective amount of inflammatory bowel disease treatment in a child with a high expression of anti-Cbir1 relative to a healthy individual.

### Increased Immune Reactivity Predicts Aggressive Complicating Crohn's Disease in Children

As disclosed herein, the inventors determined whether immune responses and/or CARD15 variants are associated with complicated disease phenotypes and predict disease progression. Sera were collected prospectively from 796 pediatric CD cases and tested for anti-Cbir1 (flagellin), anti-outer membrane protein C (anti-OmpC), anti-Saccharomyces-cerevisiae (ASCA) and perinuclear anti-neutrophil cytoplasmic antibody (pANCA) using ELISA. Genotyping (TaqmanMGB) was performed for 3 CARD15 variants (SNPs 8, 12, 13). Associations between immune responses (antibody sum (AS) and quartile sum score (QSS), CARD15, and clinical phenotype were evaluated. All phenotype assessments were performed by clinical investigators blinded to genetic and immune response analysis.

As further disclosed herein, 32% of patients developed at least one disease complication within a median of 32 months and 18% underwent surgery. 73% of patients were positive for at least 1 immune response. The frequency of IP, S and surgery significantly increased (p trend<0.0001 for all 3 outcomes) with increasing AS and QSS. 9% of seropositive groups had IP/S vs. 2.9% in the seronegative group (p=0.01). 12% of seropositive groups underwent surgery vs. 2% in the seronegative group (p=0.0001). The highest AS group (3) and QSS group (4) demonstrated the most rapid disease progression (p<0.0001). Increased hazard ratio was observed for AS group 3 (7.8 [2.2-28.7] p<0.002 and QSS group 4 (11.0 [1.5,83.0] p<0.02).

The inventors found that the rate of complicated CD increases in children as the number and magnitude of immune reactivity increases. Disease progression is significantly faster in children expressing immune reactivity. Baseline immune response assessment predict children at risk for complicating IP/S phenotypes, in whom early effective therapy would be of benefit.

In one embodiment, the present invention provides a method of predicting Crohn's Disease progression in an individual by determining the presence or absence of a high immune reactivity relative to a healthy individual. In another embodiment, the present invention provides a method of treatment of Crohn's Disease by administering a therapeutically effective amount of Crohn's Disease treatment in an individual with immune reactivity relative to a healthy individual. In another embodiment, the present invention provides a method of treating an aggressive form of Crohn's Disease in a pediatric subject by determining the presence of a high immune reactivity and treating the aggressive form of Crohn's Disease. In another embodiment, the present invention provides a method of determining the prognosis of Crohn's Disease in a subject by determining the presence or absence of a high immune reactivity relative to a child with a non-aggressive form of Crohn's Disease. In another embodiment, immune reactivity includes OmpC, ASCA, Cbir1 and/or pANCA. In another embodiment, the individual is a child. In another embodiment, the subject is a pediatric subject. In another embodiment, immune reactivity is determined by time to complication or surgery. In another embodiment, the immune reactivity is associated with disease phenotype, such as disease location, behavior and/or surgery. In another embodiment, the presence of the high immune reactivity is indicative of a prognosis of an aggressive form of Crohn's Disease.

As described herein, various embodiments provide methods of prognosis of Crohn's Disease by determining a high immune reactivity of various markers, such as OmpC, ASCA, Cbir1 and/or pANCA, where a high immune reactivity of one or more markers is associated with a prognosis of developing an aggressive form of Crohn's Disease. Immune reactivity is determined by comparing both the presence and magnitude of markers to a standard set by those marker levels found in a subject who has and maintains a non-aggressive form of Crohn's Disease.

### Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature,” Nucleic Acids Research 28:655-661 (2000)). Minor grove binders include, but are not limited to, compounds such as dihydrocyclopyrroloindole tripeptide (DPI).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Increased Immune Reactivity Predicts Aggressive Complicating Crohn's Disease in Children

Crohn's disease (CD) is a heterogeneous disorder characterized by diverse clinical phenotypes (inflammatory, fibrostenosing [FS], internal penetrating [IP]) that appear to be influenced by genetic and immune factors. Children frequently manifest an aggressive disease course, and the ability to identify those at risk for complicated disease at diagnosis would be invaluable in guiding initial therapy.

The inventors examined the association of serological immune responses and CARD15 with CD phenotype in a large well-characterized pediatric collaborative cohort. Sera were collected from 797 prospectively followed pediatric CD cases and tested for immune responses to microbial antigens: anti-Cbir1 (flagellin), anti-outer membrane protein C (anti-OmpC) and anti-Saccharomyces-cerevisiae (ASCA) using ELISA. Genotyping (TaqmanMGB) was performed for 3 CD-associated variants of CARD15 (SNPs 8, 12, 13). Disease phenotypes were determined blinded to genotype and immune responses. Associations between immune responses, CARD15 and clinical phenotype were evaluated.

CARD15 variants and immune responses were present in 34% and 78%, respectively. Small bowel (SB) location, IP and/or FS disease behavior were present in 68% (n=542) and 20% (n=152) of children after a median follow-up of 31 months. The odds of developing IP and/or FS disease were highest in patients positive for all 3 immune responses (Table 1). The highest level for each individual antibody was associated with IP and/or FS with the odds being highest when using the sum of all immune response levels (Table 2). Multivariate analysis confirmed the Anti-OmpC (p<0.0002) and anti-Cbir1 (p=0.005) association with IP as well as ASCA (p=0.02) and anti-Cbir1 (p=0.04) with FS. CARD15 was associated with small bowel disease (OR=1.7; p<0.0001) only, not with disease behavior. The rate of complicated CD increases in children as the number and magnitude of immune reactivity increases.

Baseline immune response assessment may identify children at risk for complicating IP/FS phenotypes, for whom early, aggressive immunomodulatory therapy could be of benefit.

TABLE 1
Qualitative Analysis

| | ASCA Odds Ratio | Anti-OmpC | Anti-Cbir1 | Antibody Sum (ASCA+, OMPC+, |
|---|---|---|---|---|
| | (OR); p value | OR; p value | OR; p value | Cbir1+) OR; p value |
| SB | 2.9; p < 0.0001 | NS | 1.6; p = 0.002 | 2.8; p < 0.0001 |
| FS | 2.4 p < 0.0001 | 2.7; p < 0.0001 | 2.0; p = 0.002 | 6.1; p < 0.0001 |
| IP | 2.3; p = 0.002 | 3.7; p < 0.001 | 2.3; p = 0.003 | 9.5; p < 0.0001 |

TABLE 2
Quantitative Analysis

| | ASCA | Anti-OmpC | Anti-Cbir1 | Quartile Sum |
|---|---|---|---|---|
| | OR; p value | OR; p value | OR; p value | OR; p value |
| SB | 3.5; p < 0.0001 | NS | 1.8; p = 0.003 | 3.5; p < 0.0001 |
| FS | 2.6; p = 0.0001 | 3.5; p < 0.0001 | 3.7; p < 0.0001 | 12.5; p < 0.0001 |
| IP | 2.1; p = 0.006 | 3.5; p = 0.0001 | 3.9; p = 0.002 | 8.5; p < 0.0001 |

### Example 2

### Serum Immune Responses Predict Rapid Disease Progression Among Children with Crohn's Disease: Immune Responses Predict Disease Progression

Crohn's disease (CD) is a heterogeneous disorder characterized by diverse clinical phenotypes. Childhood-onset CD has been described as a more aggressive phenotype. Genetic and immune factors may influence disease phenotype and clinical course. The inventors examined the association of immune responses to microbial antigens with disease behavior and prospectively determined the influence of immune reactivity on disease progression in pediatric CD patients.

Sera were collected from 196 pediatric CD cases and tested for immune responses: anti-12, anti-outer membrane protein C (anti-OmpC), anti-Cbir1 flagellin (anti-CBir1), and anti-Saccharomyces-cerevisiae (ASCA) using ELISA. Associations between Immune responses and clinical phenotype were evaluated.

Fifty-eight patients (28%) developed internal penetrating and/or stricturing (IP/S) disease after a median follow-up of 18 months. Both anti-OmpC (p<0.0006) and anti-12 (p<0.003) were associated with IP/S disease. The frequency of IP/S disease increased with increasing number of immune responses (p trend=0.002). The odds of developing IP/S disease were highest in patients positive for all four immune responses (OR (95% CI): 11 (1.5-80.4); p=0.03). Pediatric CD patients positive for 21 immune response progressed to IP/S disease sooner after diagnosis as compared to those negative for all immune responses (p<0.03).

The presence and magnitude of Immune responses to microbial antigens are significantly associated with more aggressive disease phenotypes among children with CO. This demonstrates that the time to develop a disease complication in children is significantly faster in the presence of immune reactivity, thereby predicting disease progression to more aggressive disease phenotypes among pediatric CD patients.

### Example 3

### Serum Immune Responses Predict Rapid Disease Progression Among Children with Crohn's Disease: Immune Responses Predict Disease Progression: Patient Population

Pediatric CD patients were enrolled from participating sites of the Western Regional Pediatric IBD Research Alliance. In order to be eligible, all CD patients must have undergone complete colonoscopy with ileal intubation or complete colonoscopy and small bowel follow through. A diagnosis of CD for this study required at least two of the following: (1) history of abdominal pain, weight loss, short stature, malaise, rectal bleeding, or diarrhea; (2) characteristic endoscopic findings of discontinuous ulcerations, cobblestoning, fistula, or severe perianal disease; (3) radiologic features of stricture, fistula, or evidence of cobblestoning, or ulceration of the mucosa; (4) macroscopic appearance at laparotomy of typical bowel wall induration, mesenteric lymphadenopathy, or serosal involvement showing creeping fat, or other inflammatory changes; (5) histopathology showing transmural inflammatory cell infiltrate or epithelial granulomas and absence of identifiable infectious agents (16). Blood for serological analysis was drawn at each of the participating sites and sent via overnight FedEx to the Genotyping Core Facility of the Medical Genetics Institute/GCRC and the Immunobiology Institute at Cedars-Sinai Medical Center (CSMC). This study was approved by the Ethics Review Board at each participating site.

### Example 4

### Serum Immune Responses Predict Rapid Disease Progression Among Children with Crohn's Disease: Immune Responses Predict Disease Progression: Data Collection

Subjects and their families completed patient demographic forms at the time of blood draw and physicians completed clinical information forms in reference to both date of diagnosis and date of last follow-up. Once collected, all data were then transferred and stored in a secure relational (Oracle) database for analysis. For the purpose of this study, key variables included date of diagnosis, age at diagnosis, date of last follow-up and duration of disease as of last follow-up, ethnicity, family history, disease location, disease behavior, granulomas, and surgical procedures.

### Example 5

### Serum Immune Responses Predict Rapid Disease Progression Among Children with Crohn's Disease: Immune Responses Predict Disease Progression: Phenotype

All phenotype assessments were performed by clinical investigators blinded to genetic and immune response analysis and based on the following uniform definitions:

Disease location at diagnosis was defined by the extent of the disease involvement at the time of initial presentation. Disease extent was based on endoscopic, histologic, and radiographic evidence of inflammation.

Disease location as of last follow-up was defined by the maximal extent of the disease involvement at the point of last follow-up or before a patient underwent first resection. Other than anal/perianal disease, location change was documented when clinically indicated investigations were performed anytime from diagnosis until the date of last follow-up. For the purpose of analysis, disease location as of last follow-up was used for all genotype/immune response-phenotype associations.

There were five disease locations that patients were categorized into (1) small bowel only: disease of the small bowel proximal to the cecum and distal to the ligament of treitz; (2) large bowel only: any colonic location between the cecum and rectum with no small bowel disease; (3) small and large bowel: disease of the small bowel and any location between the cecum and rectum; (4) upper digestive tract disease involving at least one of the following sites: esophagus, stomach, and duodenum; (5) anal: perianal and anal lesions including skin tags and anal ulcers. Patients could have been in more than one category such that patients with small and/or large bowel disease may also have concomitant upper tract and/or anal disease.

Disease behavior at diagnosis was defined by the behavior of the disease at presentation.

Disease behavior as of last follow-up was defined by the disease behavior observed as of last follow-up. At both time points, data may have been obtained after a patient underwent a surgical resection, as reliable data are often obtained at the time of surgery for defining complicated disease behaviors.

Disease behavior was divided into two broad categories: noncomplicating and complicating disease behaviors. Noncomplicating behavior referred to uncomplicated inflammatory disease without evidence of stricturing or penetrating disease behaviors (nonpenetrating nonstricturing [NPNS]). Complicating behaviors referred to penetrating and stricturing disease. (1) Stricturing disease was defined as the occurrence of constant luminal narrowing demonstrated by radiologic, endoscopic, or surgical examination combined with pre-stenotic dilatation and/or obstructive signs or symptoms. (2) Penetrating disease was defined as either IP if patients had evidence of entero-enteric or entero-vesicular fistulae, intraabdominal abscesses, or intestinal perforation, or perianal penetrating (PP) if patients developed either perianal fistulae or abscesses or recto-vaginal or ano-vaginal fistulae.

For the purpose of analysis, stricturing and IP complications were grouped into one outcome. PP and patients without complications (NPNS) comprised the other two comparison groups.

### Example 6

### Serum Immune Responses Predict Rapid Disease Progression Among Children with Crohn's Disease: Immune Responses Predict Disease Progression: Immune Responses

All blood samples were taken at the time of consent and enrollment. Sera were analyzed for expression of ASCA, antiOmpC, anti-12, and anti-CBir1 antibodies in a blinded fashion by ELISA. Analysis and IgG and IgA ASCA were performed at Cedars-Sinai Medical Center or Prometheus Laboratories using the same technology. All assays for anti-OmpC, anti-I2, and anti-CBir1 were performed at Cedars-Sinai. Antibody levels were determined and results expressed as ELISA units (EU/mL), which are relative to a Cedars-Sinai Laboratory (IgA-12, IgA-OmpC, and IgG CBir1) or a Prometheus Laboratories Standard (IgA and IgG ASCA), which is derived from a pool of patient sera with well-characterized disease found to have reactivity to this antigen.

### Example 7

### Serum Immune Responses Predict Rapid Disease Progression Among Children with Crohn's Disease: Immune Responses Predict Disease Progression: Statistical Analysis

To determine the associations between disease phenotype characteristics and antibody responses toward microbial antigens, univariate analyses using chi-squared tests were performed. Odds ratios (OR) and 95% confidence intervals were calculated to compare the odds of positive serum reactivity toward the microbial antigens (CBir1,I2. OmpC. and ASCA) in the group of patients with a certain disease characteristic with the group of patients without such a characteristic. Quantitative comparison of immune response levels between groups (IP/S+vs IP/S−) for each antibody was performed using nonparametric Wilcoxin rank test. Multivariate analysis with logistic regression modeling was also performed to determine the primary associations among qualitative serological responses with disease phenotypes. To compare the length of time to the development of a disease complication between groups, Kaplan-Meier estimator of survival probability was calculated to construct survival curves. The log-rank test was used to test if the survival curves were significantly different between subgroups of patients. All analyses were performed by using Statistical Analysis Software (Version 8.02. SAS Institute, Inc., Cary N.C.).

### Example 8

### Serum Immune Responses Predict Rapid Disease Progression Among Children with Crohn's Disease: Patient Population Results

A total of 196 pediatric CD patients were eligible for analysis. Eighty-five percent (168/196) were Caucasians and 28% were of Jewish background. The median age at diagnosis was 12 yr (1-18) and the median age at study was 13 yr (4-19). The cohort comprised 47% males and 53% females. A positive family history of IBD was reported in 29% of patients.

### Example 9

### Serum Immune Responses Predict Rapid Disease Progression Among Children with Crohn's Disease: Clinical Phenotypes Results

A total of 38 (19%) patients had either a stricturing and/or penetrating complication at the time of diagnosis. After a median follow-up time (median disease duration as of last follow-up) of 18 months (1-200), the total number of pediatric CD patients who experienced a disease complication increased to 58 (30%). Table 3 details the clinical phenotypes of the pediatric CD cohort. Of the 35 patients with internal penetrating and/or stricturing (IP/S) disease, 18 had isolated stricturing disease, 11 had IP and 6 had both complications. Thirty-two of the 58 patients (55%) underwent a combined total of 53 surgeries related to disease complications, 38 (72%) of which were small bowel surgeries for IP/S disease complications. The remaining surgeries were for perianal perforating diseases. All but two patients (15/17) with IP disease and 45% of patients with isolated stricturing disease underwent small bowel surgery as of last follow-up.

TABLE 3
Clinical Phenotypes in Pediatric CD Cohort

| Clinical Phenotype | N (%) | |
|---|---|---|
| Disease location | | |
| Small bowel only | 24 | (12.2) |
| Large bowel only | 51 | (26.0) |
| Small and large bowel | 120 | (61.2) |
| and/or upper tract | 78 | (39.8) |
| and/or anal disease | 39 | (19.9) |
| Disease behavior at diagnosis | | |
| Non-penetrating non-stricturing | 158 | (80.6) |
| Internal penetrating and/or structuring | 21 | (10.7) |
| Perianal penetrating only | 17 | (8.7) |
| Disease behavior as of last follow up | | |
| Non-penetrating non-stricturing | 138 | (70.4) |
| Internal penetrating and/or structuring | 35 | (17.9) |
| Perianal penetrating only | 23 | (11.7) |

### Example 10

### Serum Immune Responses Predict Rapid Disease Progression Among Children with Crohn's Disease: Immune Responses Results

Serum was collected at a median of 9.4 months (0-211.7) after diagnosis, 18% of patients (35/196) had serum collected at the time of diagnosis or within 1 month of diagnosis and 33% (64/196) within 3 months of diagnosis. A total of 77.0% of patients were positive for at least one immune response, 23.7% of which were positive for a combination of any two immune responses. 16.4% of patients were positive for all three responses, and 3.4% were positive for all four responses. ASCA anti-I2, anti-OmpC, and anti-CBir1 were present in 43%, 26%, 22%, and 53%, respectively.

### Example 11

### Serum Immune Responses Predict Rapid Disease Progression Among Children with Crohn's Disease: Immune Responses and CD Phenotypes Results

Presence and magnitude of immune responses influence disease behavior. A statistically significant association was not found for any of the immune responses with family history, ethnicity, or the presence of granulomas. ASCA was the only antibody significantly associated with small bowel disease location; yet was not associated with disease behavior. Both anti-I2 (p=0.0034) and anti-OmpC (p=0.0006) were associated with complicating disease behaviors, more specifically IP/S disease. The frequency of isolated perianal perforating disease was similar between immune response groups (±) for all four antibodies. In addition to the qualitative associations observed for anti-OmpC and anti-I2, the magnitude of the immune response to OmpC and I2 also had an association with internal perforation and/or stricturing disease (p=0.008 and p=0.002 for anti-OmpC and anti-I2, respectively). The anti-OmpC association continued to be significant in the multivariate logistic regression, which showed that anti-OmpC (p<0.02) was independently associated with IP/S disease. ASCA, anti-I2, and anti-Cbir1 did not show any independent association with disease behavior.

Cumulative influence of immune responses on disease behavior. Individually there is a clear association with individual immune responses I2 and OmpC with IP/S. The inventors then examined whether there was a cumulative influence of immune responses on disease behavior and determined if the odds of having complicating IP/S disease were greater in the presence of multiple immune responses. As demonstrated, the frequency of IP/S disease significantly increased (p trend=0.002) as the number of immune responses increased. The OR demonstrate that the odds of having IP/S disease was significantly increased in children positive for a combination of any three immune responses (OR [95° CI]; OR=5.5 [1.3-23.6]; p=0.02) and even more so in children positive for all four immune responses (OR [95% CI]; OR=11.0 [1.5-80.4]; p=0.03) as compared to those patients negative for all immune responses (baseline group).

### Example 12

### Serum Immune Responses Predict Rapid Disease Progression Among Children with Crohn's Disease: Disease Progression Results

Based on the cross-sectional data, immune responses are associated with the presence of disease complications. For the second aim of the study, the inventors set out to examine whether seropositive patients (≧1 immune response) have a greater risk to progress to IP/S as compared to seronegative patients (0 immune responses). The inventors used a longitudinal study to answer this question which included only those patients who did not have IP/S at diagnosis (NPNS±PP) and continued to be uncomplicated (NPNS±PP) at the time the serum was collected for immune response measurement so that we could be certain that when clinically recognizable IP/S occurred it was after the sera were collected for antibody measurement. The median time from diagnosis to serum draw was 9.2 months (0-142.3). Among those who developed IP/S (10/167) during the follow-up, the median time from diagnosis to the onset of IP/S was 48 months. As of last follow-up, 8.2% (8/97) of the seropositive group had IP/S versus only 2.9% (2/70) in the seronegative group. Because longer disease duration increases the chance of developing IP/S and not all patients are followed for the same amount of time, the inventors performed survival analysis to take the length of follow-up into consideration. The inventors first evaluated survival with OmpC, I2, and ASCA. Given the same length of follow-up, among those patients positive for at least one serology, more progressed to IP/S than those negative for the three serologies (p=0.03). Saying it differently, those patients positive for at least one serology progressed to IP/S faster than those negative for all three serologies. We then examined whether the addition of Cbir1 changed the survival outcome. Of significance is that the two patients who developed IP/S in the presumptive seronegative group, when measuring 12, OmpC, and ASCA only, were actually CBir1 positive. The inventors have fewer patients followed out long enough in those who had all four antibodies measured. Thus, when the inventors have adequate such numbers these anti-CBIR positive patients would be reclassified to the seropositive group. As of last follow-up, all seronegative patients remained complication free.

### Example 13

### Serum Immune Responses Predict Rapid Disease Progression Among Children with Crohn's Disease: Conclusion

The inventors have demonstrated that immune reactivity to specific microbial antigens is associated with complicating disease behaviors. This study demonstrates that immune responses to an increasing number of microbial antigens are associated with complicating IP/S disease behaviors in pediatric CD patients. Moreover, disease progression to a more aggressive disease phenotype in children is accelerated in the presence of immune reactivity. Serum immune responses predict a more rapid disease progression from uncomplicated to complicated disease.

### Example 14

### CARD8: A Novel Association with Childhood-Onset Ulcerative Colitis (UC)

CARD proteins play an important role in apoptosis and cytokine regulation, including NfKB, processes which are important in the pathogenesis of IBD. CARD15/NOD2 was the first novel gene reported to confer Crohn's disease (CD) susceptibility and influence disease phenotype. CARD4 has not been found to be associated with CD. McGovern et al reported a significant CD association with the CARD8/TUCAN/CARDINAL gene toured at 19q13.3 in adult patients.

The inventors investigated the association of the CARD8-T10C polymorphism with susceptibility to UC and CD in children. DNA was collected from 342 subjects (75 CD trios, 39 UC trios). Both parents and the affected child were genotyped for 3 allelic variants of the CARD15 gene (R702W. G908R, 1007insC, also referred to as SNP 8, 12 and 13) as an association control and 1 variant of the CARD gene (T10C) using Taqman technology. The transmission disequilibrium test (TDT) was used to test association with either UC or CD using GENEHUNTER 2.0.

CARD8 allele T was present in 63% of CD patients and 77% of UC patients. CARD15 frequency (any variant) was 25% and 11% in CD and in UC, respectively. Similar frequencies were observed for parents for both genes. As expected, transmission distortion was seen for all CARD15 variants in CD, but not in UC. No association was observed between CARD8 and CD, however, in contrast, TDT showed a highly significant association with UC, with over transmission of the CARD8 common allele (Table 4).

This shows a CARD8 association with childhood-onset UC. The over transmission of the common allele in this analysis is similar to that which is seen with PPARgamma in type 2 diabetes and the insulin gene polymorphism in type 1 diabetes. These findings are in contrast to the adult CD association showing different mechanisms for pediatric IBD.

TABLE 4
TDT Analysis.

| | CARD8 T allele | | | CARD13 SNP 8, 12.13 | | |
|---|---|---|---|---|---|---|
| | | NOT | | | NOT | |
| | TRANSMITTED | TRANSMITTED | pvalue | TRANSMITTED | TRANSMITTED | pvalue |
| CD | 37 | 33 | NS | 30 | 21 | 0.003 |
| (a = 75) | | | | | | |
| UC | 23 | 8 | 0.007 | 4 | 7 | NS |
| (n = 39) | | | | | | |

### Example 15

### Antibodies to a Novel Flagellin (CBIR1) Adds Clinical Utility to the Diagnosis and Differentiation of Pediatric IBD

Approximately ⅔ of IBD patients are positive for antibodies to microbial and auto-antigens. A novel antibody, anti-Cbir1, may have unique diagnostic properties and phenotypic associations in children. The inventors examined the added utility of anti-Cbir1 in the diagnosis and differentiation of pediatric IBD patients as compared to previously defined antibodies: ASCA, OmpC, 12 and pANCA.

Sera from 331 pediatric IBD patients (111 UC, 220 CD) were tested by ELISA for anti-OmpC, anti-12, ASCA, anti-Cbir1 and pANCA. Quantitative and qualitative expression of antibody markers was evaluated. Anti-Cbir1 was present in 55% of CD vs. 15% of UC (p<0.001). 41% of anti-Cbir1 (+) UC patients were also positive for >I CD-related antibody. Anti-Cbir1 was present in 53% of ASCA(−) CD patients and in 52% (31/60) of patients negative for all antibodies. The most Cbir1 reactive CD subset was OmpC+/I2+ (74% median=49) and least reactive was ASCA+ (56%, median=31). 13.5% of pANCA (+) only UC patients were anti-CBir1 (+) as compared to 35% of pANCA(+) only CD patients (p=0.03). Both pANCA and anti-Cbir1 levels were higher in pANCA (+) CD vs. UC (median pANCA: 46.6 vs. 70.0: p=0.003, and median anti-Cbir1: 21 vs. 12 p<0.0001).

Anti-Cbir1 increased detection of CD cases negative for all other antibodies. Cbir1 reactivity added to the differentiation of pANCA+ CD from pANCA+ UC and can minimize misdiagnosed CD colitis patients. Both the presence and magnitude of anti-Cbir1 reactivity adds to the clinical utility of presently known antibodies in pediatric IBD.

### Example 16

### Increased Immune Reactivity Predicts Aggressive Complicating Crohn's Disease in Children

The inventors determined whether immune responses and/or CARD15 variants are associated with complicated disease phenotypes and predict disease progression. Sera were collected prospectively from 796 pediatric CD cases and tested for anti-Cbir1 (flagellin), anti-outer membrane protein C (anti-OmpC), anti-Saccharomyces-cerevisiae (ASCA) and perinuclear anti-neutrophil cytoplasmic antibody (pANCA) using ELISA. Genotyping (TaqmanMGB) was performed for 3 CARD15 variants (SNPs 8, 12, 13). Associations between immune responses (antibody sum (AS) and quartile sum score (QSS), CARD15, and clinical phenotype were evaluated.

32% of patients developed at least one disease complication within a median of 32 months and 18% underwent surgery. 73% of patients were positive for at least 1 immune response. The frequency of IP, S and surgery significantly increased (p trend<0.0001 for all 3 outcomes) with increasing AS and QSS. 9% of seropositive groups had IP/S vs. 2.9% in the seronegative group (p=0.01). 12% of seropositive groups underwent surgery vs. 2% in the seronegative group (p=0.0001). The highest AS group and QSS group demonstrated the most rapid disease progression (p<0.0001). Increased hazard ratio was observed for AS group 3 (7.8 [2.2-28.7] p<0.002 and QSS group 4 (11.0 [1.5,83.0] p<0.02).

The inventors found that the rate of complicated CD increases in children as the number and magnitude of immune reactivity increases. Disease progression is significantly faster in children expressing immune reactivity. Baseline immune response assessment predict children at risk for complicating IP/S phenotypes, in whom early effective therapy would be of benefit.

### Example 17

### Increased Immune Reactivity Predicts Aggressive Complicating Crohn's Disease in Children: Patient Population

Pediatric CD patients were enrolled from 21 participating sites of the Western Regional Pediatric IBD Research Alliance, The Pediatric IBD Collaborative Research Group and the Wisconsin Pediatric IBD Alliance.

In order for pediatric CD patients to be eligible, all CD patients must have undergone complete colonoscopy with ileal intubation or complete colonoscopy and small bowel follow through. A diagnosis of CD was based on standard diagnostic criteria. Blood for serological analysis was drawn and sent to The Immunobiology Institute at Cedars-Sinai Medical Center (CSMC) for all sites in the Western Regional and Wisconsin Alliance. Serological analyses were run at Prometheus Laboratories (San Diego, Calif.) for all patients drawn at sites of the Pediatric IBD Collaborative Research Group. Genotyping was performed by the Genotyping Core Facility of the Medical Genetics Institute/GCRC at CSMC for all Western Regional sites, at the Children's Hospital of Wisconsin (SK) for the Wisconsin Alliance, and at Prometheus Laboratories for all sites of The Pediatric IBD Collaborative Research Group.

### Example 18

### Increased Immune Reactivity Predicts Aggressive Complicating Crohn's Disease in Children: Disease Location

Disease location was defined by the extent of the disease involvement at the time of initial presentation. Disease extent was based on endoscopic, histologic and radiographic evidence of inflammation.

There were 5 disease locations that patients were categorized into: 1) Small bowel only: disease of the small bowel proximal to the cecum and distal to the ligament of treitz; 2) Large bowel only: any colonic location between cecum and rectum with no small bowel disease; 3) Small and large bowel: disease of the small bowel and any location between cecum and rectum; 4) Upper digestive tract: disease involving at least one of the following sites: esophagus, stomach, duodenum; 5) Anal: perianal and anal lesions including skin tags and anal ulcers. Patients could have been in more than one category such that patients with small and/or large bowel disease may also have concomitant upper tract and/or anal disease.

### Example 19

### Increased Immune Reactivity Predicts Aggressive Complicating Crohn's Disease in Children: Disease Behavior

Disease behavior at diagnosis was defined by the behavior of the disease at presentation. Disease behavior as of last follow-up was defined by the disease behavior observed as of last follow-up. At both time points, data may have been obtained after a patient underwent a surgical resection, as reliable data is often obtained at the time of surgery for defining complicated disease behaviors.

Disease behavior was divided into 2 broad categories: non-complicating and complicating disease behaviors: non-complicating behavior: referred to uncomplicated inflammatory disease without evidence of stricturing or penetrating disease behaviors (non-stricturing non-penetrating [NPNS]). Complicating behaviors referred to penetrating and stricturing disease. 1) Stricturing disease (S): was defined as the occurrence of constant luminal narrowing demonstrated by radiologic, endoscopic or surgical examination combined with pre-stenotic dilatation and/or obstructive signs or symptoms. 2) Penetrating disease: was defined as either internal penetrating (IP) if patients had evidence of entero-enteric or entero-vesicular fistulae, intra-abdominal abscesses or intestinal perforation or perianal penetrating (PP) if patients developed either perianal fistulae or abscesses or recto-vaginal or ano-vaginal fistulae.

### Example 20

### Increased Immune Reactivity Predicts Aggressive Complicating Crohn's Disease in Children: Immune Responses

All blood samples were taken at the time of consent and enrollment. Sera were analyzed for expression of pANCA, ASCA, anti-OmpC, and anti-CBir1 antibodies in a blinded fashion by ELISA. Serological analyses were performed at CSMC or Prometheus Laboratories using the same technology. Antibody levels were determined and results expressed as ELISA units (EU/ml), which are relative to a Cedars-Sinai Laboratory or a Prometheus Laboratories Standard which is derived from a pool of patient sera with well-characterized disease found to have reactivity to this antigen.

### Example 21

### Increased Immune Reactivity Predicts Aggressive Complicating Crohn's Disease in Children: Definitions of Immune Responses

The following definitions were used for all analyses involving ASCA, anti-OmpC and anti-CBir1 immune responses. pANCA was analyzed separately given that pANCA has been shown to be negatively associated with the majority of disease phenotypes except large bowel disease location.

Antibody sum (AS): number of positive antibodies per individual: 0, or 1 or 2, or 3 positive.

Antibody Quartile Score: quartile score for each antibody level (<25%=1, 25-50%=2, 51%-<75%=3, 75%-100%=4).

Quartile Sum Score (QSS): sum of quartiles score for all 3 antibodies (ASCA (A or G, anti-OmpC and anti-CBir1). Minimum score of 3 (all antibodies had a quartile score of 1) and maximum score of 12 (all antibodies had a quartile score of 4).

Quartile Sum Score (QSS) Group: In order to minimize the number of patient subsets i.e quartile sum score 3-12, the inventors regrouped patients based on a range of quartile sum scores: Quartile sum score 3-5=group 1, 6-7=group 2, 8-9 group 3 and 10-12=group 4.

### Example 22

### Increased Immune Reactivity Predicts Aggressive Complicating Crohn's Disease in Children: Genotyping

Three single nucleotide polymorphisms (SNP's) in the CARD15 gene have been associated with CD. CARD15 SNP's R675W (rs2066844, CEPH-IBD1-snp8), G881R (rs2066845, CEPH-IBD1-snp12), and 3020insC (rs2066847, CEPH-IBD1-snp13) were adapted to the TaqMan MGB genotyping platform following the manufacturer's instructions and using PrimerExpress design software (Applied Biosystems, Foster City, Calif.). The TaqMan MGB platform is a two-probe, 5′-exonuclease PCR assay that employs a minor groove binder on the 3′-end of the probes in order to give greater allele discrimination.

### Example 23

### Increased Immune Reactivity Predicts Aggressive Complicating Crohn's Disease in Children: Statistical Analysis

To determine the associations between disease phenotype characteristics and antibody responses toward microbial antigens, univariate analyses using χ² tests were performed. Odds ratios (OR) and 95% confidence intervals were calculated to compare the odds of positive serum reactivity (antibody sum, quartile sum score, quartile sum score group) towards the microbial antigens (CBir1, OmpC, and ASCA) in the group of patients with a certain disease characteristic with the group of patients without such a characteristic. For the OR calculations the minimum antibody sum of 0, the minimum quartile sum score of 3 and the minimal quartile sum score group 1 were set as baseline, i.e. OR of 1.0 Quantitative comparison of immune response levels between groups (IP/S+vs. IP/S−) for each antibody was performed using non-parametric Wilcoxin Rank test. Stepwise multivariable analysis using logistic regression modeling was also performed to determine the primary associations among qualitative serological responses with disease phenotypes. To compare the length of time to the development of a disease complication between groups, Kaplan-Meier estimator of survival probability was calculated to construct survival curves. The log-rank test was used to test if the survival curves were significantly different between subgroups of patients. The hazard ratio (HR) of occurrence of complication or surgery among patients who were sera positive compared to those who were sera negative as well as who were in higher antibody sum or quartile sum group compared to those who were in baseline group were estimated from Cox's proportional hazards model and adjusted for all other covariates. All HRs were expressed as a point estimate with 95% confidence interval. Patients who only had sera data after the occurrence of complications or surgery were not included in the survival analysis. Age at diagnosis and gender were included as covariates in all the multivariable analyses. The OR/HR for age at diagnosis was explained as the times of odds/hazards increase (e.g. OR-1) per one year older at diagnosis. All analyses were performed by using Statistical Analysis Software (Version 9.1; SAS Institute, Inc., Cary, N.C.).

### Example 24

### Increased Immune Reactivity Predicts Aggressive Complicating Crohn's Disease in Children: Patient Demographics

A total of 796 pediatric CD patients were eligible for analysis. Eighty-seven percent (694/796) were Caucasians and 28% were of Jewish background. The median age at diagnosis was 12 [0.6-18] years and the median disease duration as of last follow up was of 32 [1-235] months. The cohort was comprised of 56% males and 44% females.

### Example 25

### Increased Immune Reactivity Predicts Aggressive Complicating Crohn's Disease in Children: Clinical Phenotypes

A total of 236 (30.3%) patients presented with (96/796 [12%]) or developed (140/796 [18%]) at least one disease complication within the median follow up time of 32 months: 116 stricturing disease, 70 internal penetrating, and 115 perianal penetrating disease. Ten patients had all 3 complications and 45 had a combination of 2 of the 3 complications. One hundred and forty patients (18%) underwent a CD related surgery of which 89 were small bowel resections. Of the remaining surgeries: a total of 42 were involving perianal penetrating disease; 24 patients underwent colectomy and 3 patients a limited colonic resection. Fifteen patients had more than one surgery.

### Example 26

### Increased Immune Reactivity Predicts Aggressive Complicating Crohn's Disease in Children: Immune Response and Genotype Frequencies

Serum was collected at the time of diagnosis or within 1 month of diagnosis in 18% (146/796) of patients and 30% (241/796) within 3 months of diagnosis. The remaining patients had serum collected greater than 3 months from time of diagnosis. A total 73% of patients were positive for at least one microbial driven immune response (ASCA, anti-OmpC or anti-Cbir1), 27% of whom were positive for a combination of any 2 of these immune responses and 8% of patients were positive for all 3 responses. ASCA, anti-OmpC, anti-CBir1 and pANCA were present in 45%, 18%, 52%, and 19% respectively. NOD2/CARD15 (any variant) was observed in 34% of patients (25% heterozygote and 9% homozygote or compound heterozygote).

### Example 27

### Increased Immune Reactivity Predicts Aggressive Complicating Crohn's Disease in Children: Cross Sectional Analyses

Univariate analysis of immune responses and NOD2/CARD15 genotype demonstrated that NOD2/CARD15 (all variants individually or any variant) was only associated with small bowel disease location (OR [95% CI] 1.9 [1.4-2.7] p<0.0001) and had no association with disease behavior. ASCA was associated with small bowel disease (2.9 [2.1-4.0] P<0.0001) and perianal disease (1.5 [1.1-2.2]<0.02). C Bir1 was also associated with small bowel disease (1.6 [1.2-2.3] p=0.002) and OmpC had no significant association with any disease location. pANCA was associated with large bowel disease (4.0 [1.8-8.8] p<0.0001). ASCA, anti-CBir1 and anti-OmpC were negatively associated with non-penetrating non stricturing disease (NPNS); in contrast all showed a positive association with complicating disease and surgery. The odds of having internal penetrating (IP), perianal penetrating (PP), stricturing (S) disease and surgery were highest in the presence of anti-OmpC. As disclosed herein, there was a cumulative influence of number of immune responses (antibody sum) as well as the magnitude of the immune response (quartile sum score group) on disease behavior. The frequency of internal penetrating, stricturing disease and surgery significantly increased (p trend<0.0001) as the number of immune responses increased (antibody sum 0-3) and magnitude of immune response (quartile sum score group 1-4) increased. The odds ratios for the 3 disease behaviors and surgery associated with antibody sum and quartile sum score groups are disclosed herein.

Multivariable analysis confirmed the association of small bowel location with ASCA (OR [95% CI]: 2.3 [1.6-3.2]; p<0.0001), anti-CBir1 (OR 1.5 [1-1.2]; p=0.03), pANCA (OR: 0.6 [0.4-0.9]; p=<0.007); and NOD2/CARD15 (OR; 1.7 [1.1-2.4]; p=0.007). Large bowel location was associated with pANCA (OR: 2.8 [1.4-5.4]; p<0.004). Results of the multivariable analysis for the independent associations with disease behavior and surgery are disclosed herein. All individual antibodies were included in the model as well as a single unit change in antibody quartile sum score as a co-variable (e.g. increase in score of 3 to 4). There was a significant association seen with quartile sum score change and complicating disease behaviors as well as surgery, such that for each unit of quartile sum increase the OR increased by 1.3 for internal penetrating and stricturing disease and 1.2 for surgery. The difference between a score of the minimum 3 and the maximum score of 12 equates to an OR of 10.6 (=1.3)⁹ and 5.2 (=1.2)⁹, respectively. Quartile sum score was not independently associated with small bowel disease location as compared to the presence of the individual antibodies as noted above. These results show that disease location is associated more so with the presence of the immune responses and less so by the antibody levels, whilst disease behavior and surgery are more significantly associated with the magnitude of the immune response. Additional independent associations were found between female gender and older age at diagnosis.

### Example 28

### Increased Immune Reactivity Predicts Aggressive Complicating Crohn's Disease in Children: Predictors of Disease Progression

The inventors' cross-sectional data demonstrate that both single and multiple immune responses are associated with the presence of disease complications and surgery. For the second aim of the study, the inventors set out to examine whether seropositive patients (1, 2, or 3 positive for ASCA, anti-OmpC and/or anti-CBir1) had a greater risk to progress to internal penetrating and/or stricturing (IP/S) disease as well as to surgery, as compared to seronegative patients (0 such immune responses). The inventors used a longitudinal study to answer this question which included only those patients who did not have IP/S or surgery at diagnosis (NPNS+/−PP) and continued to have uncomplicated disease status at the time the serum was collected for immune response measurement. Thus the inventors could be certain that in these individuals, when clinically recognizable IP/S or surgery occurred, it did so after the serum was collected. A total of 536 patients met these inclusion criteria. The median time from diagnosis to serum draw was 10 [0-211] months for the 536 patients included in the prospective analysis. A total of 90 of the entire prospective cohort of patients (n=536) developed IP/S in follow up; however 59% (53 patients) were eliminated from this analysis as they had immune responses collected after the complication occurred. Among the 37 patients who developed IP/S during the follow-up after serum was drawn, the median [range] time from diagnosis to the onset of IP/S was 26 [4-108] months. Thirty two of the 363 seropositive patients (9%) had IP/S vs. only 2.9% (5/173). in the seronegative group (p=0.01). Among the 61 patients who underwent surgery (any CD related surgery after serum was drawn) the median [range] time from diagnosis to surgery was 30 [1-105] months. Twelve percent (57/464) of the seropositive (at least one positive) patients had undergone surgery vs. only 2% (4/189) in the seronegative group (p=0.0001). Because longer disease duration increases the chance of developing IP/S as well as surgery, and not all patients were followed for the same amount of time, we performed survival analysis to take the length of follow-up into consideration. The Kaplan-Meier survival analysis, followed by the log-rank test for the different antibody sum and quartile sum score group comparisons, showed that overall survival times for IP/S and CD-related surgery were significantly lower for those positive for immune responses, and this was true when both the quantity of immune responses and magnitude of those responses were assessed. The first analyses examined antibody sum: 0 vs. 1 vs. 2 vs. 3 and time to development of IP and/or S as well as time to surgery. Given the same length of follow up, among those patients with antibody sum greater than 1, more progressed to IP/S than those negative for all 3 or positive for only 1 antibody (p=<0.0001). In other words, those patients positive for at least 2 immune responses (antibody sum 2 or 3) progressed to IP/S faster that those negative for all or positive for only 1 antibody. The group positive for all 3 antibodies demonstrated the most rapid disease progression with a median [range] time to disease progression of 20 [4-65] months. The same rapid progression to surgery was seen among the higher antibody sum group. Like antibody sum, those patients in the highest quartile sum score group (group 4=Quartile sum score 10-12) progressed faster to IP/S and surgery and the median [range] time to IP/S and surgery was 21 [4-65] months and 27 [1-93] months, respectively. The survival curves were very similar when evaluating intestinal resection only (n=48) as compared to any CD surgery (n=61) (Log Rank: p<0.0001 for the 4 antibody sum groups and p=0.001 when comparing survival among the 4 quartile sum groups). The most conservative way to evaluate the predictive abilities of immune response was to limit inclusion in the survival analysis to only patients whose serum was drawn before a complication or surgery. The inventors also performed survival analysis on all 90 patients who developed IP and/or S in follow up regardless of when serologies were drawn. For both antibody sum and quartile sum score group, the results showed a significantly higher number of patients progressing to complication faster in the face of seropositivity.

The predictive ability of immune responses for rapid progression to the first IP/S or surgical event was further evaluated by fitting Cox-proportional hazards models. OmpC (HR [95% CI]; p value) (2.4[1.24.9]; p=0.01) and CBir1 (2.5[1.2-5.2]; p<0.02), but not ASCA, were associated with increased hazard of IP/S, as was older age at diagnosis (1.2 [1.1-1.3]; p=0.004). Lower hazards were observed with pANCA positivity (0.16 [0.04-0.70]; p<0.02). Antibody sums 2 and 3 as well as quartile sum score groups 3 and 4 were associated with an increased hazard for developing disease complications (IP/S). Hazard Ratios for all CD related surgeries as well as for intestinal resections only were calculated controlling for both disease location and disease complication (IP, S and PP). OmpC was associated with increased hazard of any CD related surgery (2.2 [1.3-3.8;] p=0.004 or intestinal resection surgery (3.5 [1.9-6.4]; p=0.001). The Cox proportional hazard model also tested the predictive ability of antibody sum groups and quartile sum score groups for surgery. Results of any CD related surgery are disclosed herein. When examining intestinal resection surgery, an increased hazard was observed for antibody sum 3 (7.8 [2.2-28.7]; p<0.002 and quartile sum score group 4 (11.0 [1.5-83.0]; p<0.02).

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

1. A method of diagnosing susceptibility to a subtype of Crohn's Disease in a child, comprising:
determining the presence or absence of at least one risk variant at the CARD15 locus selected from the group consisting of SNP8, SNP12, and SNP13, and
determining the presence or absence of at least one risk serological marker, selected from the group consisting of Cbir1, OmpC, and ASCA,
wherein the presence of at least one variant and at least one risk serological marker is diagnostic of susceptibility to the subtype of Crohn's Disease in a child.

2. The method of claim 1, wherein the subtype of Crohn's Disease in a child comprises an aggressive complicating phenotype.

3. The method of claim 1, wherein the subtype of Crohn's Disease in a child comprises a small bowel disease phenotype.

4. The method of claim 1, wherein the subtype of Crohn's Disease in a child comprises an internal penetrating and/or fibrostenosing disease phenotype.

5. The method of claim 1, wherein the presence of three of said risk serological markers presents a greater susceptibility than the presence of two, one or none of said risk serological markers, and the presence of two of said risk serological markers presents a greater susceptibility than the presence of one or none of said risk serological markers but less than the presence of three of risk serological markers, and the presence of one of said risk serological markers presents a greater susceptibility than the presence of none of said risk serological markers but less than the presence of three or two of said risk serological markers.

6. The method of claim 1, wherein the SNP8 comprises SEQ. ID. NO.: 2.

7. The method of claim 1, wherein the SNP12 comprises SEQ. ID. NO.: 3.

8. The method of claim 1, wherein the SNP13 comprises SEQ. ID. NO. 4.

9. A method of diagnosing susceptibility to a subtype of Crohn's Disease in a child, comprising:
determining the presence or absence of a high immune reactivity relative to a healthy individual for at least one risk serological marker, selected from the group consisting of Cbir1, OmpC, ASCA, I2, and pANCA,
wherein the presence of a high immune reactivity relative to a healthy individual to at least one risk serological marker is diagnostic of susceptibility to the subtype of Crohn's Disease in a child.

10. The method of claim 9, wherein the subtype of Crohn's Disease in a child comprises an aggressive complicating phenotype.

11. The method of claim 9, wherein a high immune reactivity comprises a high magnitude of expression for the risk serological marker.

12. The method of claim 9, wherein the presence of four of said risk serological markers presents a greater susceptibility than the presence of three, two, one or none of said risk serological markers, and the presence of three of said risk serological markers presents a greater susceptibility than the presence of two, one or none of said risk serological markers but less than the presence of four of said risk serological markers, and the presence of two of said risk serological markers presents a greater susceptibility than the presence of one or none of said risk serological markers but less than the presence of four or three of said risk serological markers, and the presence of one of said risk serological markers presents a greater susceptibility than the presence of none of said risk serological markers but less than the presence of four or three or two of said risk serological markers.

13. A method of treating Crohn's Disease in a child, comprising determining the presence of a high immune reactivity to a risk serological marker relative to a healthy individual, and administering a therapeutically effective amount of Crohn's Disease treatment.

14. A method of diagnosing ulcerative colitis in an individual, comprising determining the presence or absence of a risk variant at the CARD8 locus, wherein the presence of the risk variant at the CARD8 locus is diagnostic of susceptibility to ulcerative colitis.

15. The method of claim 14, wherein the risk variant at the CARD8 locus comprises SEQ. ID. NO.: 6.

16. The method of claim 14, wherein the individual is a child.

17. A method of determining the prognosis of Crohn's Disease in an individual, comprising:
determining the presence or absence of a high immune reactivity relative to a healthy individual for at least one risk serological marker, selected from the group consisting of Cbir1, OmpC, ASCA, and pANCA,
wherein the presence of a high immune reactivity relative to a healthy individual to at least one risk serological marker is indicative of a prognosis of an aggressive form of Crohn's Disease.

18. The method of claim 17, wherein the individual is a child.

19. The method of claim 17, wherein the prognosis of an aggressive form of Crohn's Disease further comprises a rapid complicating internal penetrating and/or fibrostenosing disease phenotype.

20. A method of determining the prognosis of Crohn's Disease in a pediatric subject, comprising:
determining the presence or absence of a high immune reactivity of Cbir1, OmpC, ASCA, and pANCA in the pediatric subject relative to a child who has and maintains a non-aggressive form of Crohn's Disease,
wherein the presence of the high immune reactivity relative to a child who has and maintains a non-aggressive form of Crohn's Disease is indicative of a prognosis of an aggressive form of Crohn's Disease in the pediatric subject.

21. The method of claim 20, wherein the aggressive form of Crohn's Disease further comprises a rapid complicating internal penetrating and/or stricturing disease phenotype.

22. A method of treating an aggressive form of Crohn's Disease in a pediatric subject, comprising:
determining the presence of a high immune reactivity of Cbir1, OmpC, ASCA and pANCA relative to a child who has and maintains a non-aggressive form of Crohn's Disease to prognose the aggressive form of Crohn's Disease; and
treating the aggressive form of Crohn's Disease.

23. A method of determining the prognosis of Crohn's Disease in a subject, comprising:
determining the presence or absence of a high immune reactivity in the subject relative to an individual who has and maintains a non-aggressive form of Crohn's Disease for at least one risk serological marker, selected from the group consisting of Cbir1, OmpC, ASCA, and pANCA,
wherein the presence of the high immune reactivity relative to an individual who has and maintains a non-aggressive form of Crohn's Disease is indicative of a prognosis of an aggressive form of Crohn's Disease.

24. The method of claim 23, wherein the subject is a pediatric subject.

25. The method of claim 23, wherein the individual who has and maintains a non-aggressive form of Crohn's Disease is a child.

26. The method of claim 23, wherein the aggressive form of Crohn's Disease further comprises a rapid complicating internal penetrating and/or fibrostenosing disease phenotype.

27. A method of treating an aggressive form of Crohn's Disease in a subject, comprising:
determining the presence of a high immune reactivity relative to an individual who has and maintains a non-aggressive form of Crohn's Disease to prognose the aggressive form of Crohn's Disease; and
treating the aggressive form of Crohn's Disease.

28. The method of claim 27, wherein the subject is a pediatric subject.

29. The method of claim 27, wherein the individual who has and maintains a non-aggressive form of Crohn's Disease is a child.

30. The method of claim 27, wherein the aggressive form of Crohn's Disease further comprises a rapid complicating internal penetrating and/or fibrostenosing disease phenotype.
